(19) United States
(12) Patent Application Publication
(10) Pub. No.: US 2025/0216406 A1
(43) Pub. Date: Jul. 3, 2025

(54) A SENSOR FOR TESTING BIOMARKERS IN NANO LITRE VOLUMES OF PLASMA BASED ON LUMINESCENCE

(71) Applicant: ENZYRE BV, Nijmegen (NL)

(72) Inventors: Waander VAN HEERDE, Nijmegen (NL); Mark VAN GEFFEN, Nijmegen (NL); Rachel VESTERING, Nijmegen (NL); Lisa VAN ENGELSHOVEN, Nijmegen (NL); Danique STEEGHS, Nijmegen (NL); Kees VAN 'T VEER, Nijmegen (NL); Steven KUIJPERS, Nijmegen (NL)

(21) Appl. No.: 18/851,497

(22) PCT Filed: Mar. 29, 2023
(86) PCT No.: PCT/EP2023/058191

§ 371 Date: Sep. 26, 2024

(30) Foreign Application Priority Data
Mar. 29, 2022 (GB) 2204431.7

(51) Int. Cl. G01N 33/86; B01L 3/00; G01N 21/76

(52) U.S. Cl. CPC G01N 33/86; B01L 3/502753; G01N 21/76; B01L 2200/16; B01L 2300/0663; B01L 2300/0681; B01L 2300/1805; G01N 2201/12

(57) ABSTRACT

A sensor for sensing photons in liquids comprises: a microfluidic substrate comprising a blood inlet, a buffer inlet, a mixing region configured to mix blood with buffer to provide a sample, and a plurality of microfluidic detection chambers each configured to receive a volume of analyte of less than 5 micro litres, wherein the microfluidic substrate is nontransparent and has a planar face. The sensor also comprises a semiconductor substrate comprising a plurality of single photon avalanche photodiodes facing the planar face of the microfluidic substrate and arranged in a plurality of primary arrays of photodiodes and at least one secondary array of photodiodes. The sensor also comprises a plurality of sensing channels, wherein each sensing channel of the plurality of sensing channels comprises one of the microfluidic detection chambers and a corresponding one of the primary arrays of photodiodes. Each one of the photodiodes in the secondary array of photodiodes is covered to prevent ingress of light and a secondary charge count derived from the secondary array of photodiodes is indicative of a dark count. An adjusted charge count is provided by using the dark count value to adjust the primary charge count.

### BACKGROUND

Blood coagulation activity assays usually require a volume of citrated blood of at least 0.5 mL, often 2 mL, and even 5 to 10 mL. Such volumes must be withdrawn from the subject by a phlebotomy performed by a trained technician, which is invasive and sometimes painful. Chronic patients, such as haemophilia patients, have been known to gain calluses and scar formation on the site of blood withdrawal due to the numerous procedures performed in time. One reason for the requirement for volumes of at least 0.5 mL is that the assays are performed with plasma using standard lab equipment with relatively large dead/void volumes. Also, standard commercial blood tubes generally have a volume of 1 mL or 2 mL, and not generally smaller.

After phlebotomy the blood tube is centrifugated to collect plasma by the trained technician. Depending on the assay and the number of assays, at least 0.5 mL and often 1 mL of citrated plasma is inserted in platform, of which ultimately a smaller portion of the plasma (perhaps 100 μl or even less) is diluted by the platform for the specific blood coagulation analysis. For chronic patients and especially young children, the process of providing the blood sample by phlebotomy by a trained technician represents a physical and/or psychological burden. Also, the volume of blood required is often too large to obtain from neonatal children.

A reduction in the amount of blood required would be advantageous for all of these reasons, as well as for the fact that a reduced volume requirement would be less invasive for the subject.

### SUMMARY

Against this background there is provided a sensor for sensing photons in liquids. The microfluidic substrate is capable of processing a blood plasma together with buffer and reagent into chambers having volumes of less than 5 μL, or less than 2 μL or less than 1 μL or less than 0.5 μL, or less than 0.2 μL. In one embodiment, the sensor may be capable of analysing a volume of liquid that is 0.44 μL. In another embodiment, the sensor may be capable of analysing a volume of liquid that is 0.11 μL.

The sensor comprises:
a microfluidic substrate comprising a blood inlet, a buffer inlet, a mixing region configured to mix blood with buffer to provide a sample, and a plurality of microfluidic detection chambers each configured to receive a volume of analyte of less than 5 micro litres, wherein the microfluidic substrate is non-transparent such as to prevent photons from passing through the microfluidic substrate, wherein the microfluidic substrate comprises a planar face and each microfluidic detection chamber comprises a transparent aperture located in the planar face;
a semiconductor substrate comprising a plurality of single photon avalanche photodiodes facing the planar face of the microfluidic substrate and arranged in a plurality of primary arrays of photodiodes and at least one secondary array of photodiodes,
a plurality of sensing channels, wherein each sensing channel of the plurality of sensing channels comprises one of the microfluidic detection chambers and a corresponding one of the primary arrays of photodiodes arranged such that each photodiode in the primary array is aligned with and no more than 1,000 micrometres from its corresponding microfluidic detection chamber so as to receive photons emitted within the corresponding microfluidic detection chamber, wherein a primary charge count derived from the primary array of photodiodes provides a pre-adjusted value of photon count indicative of a total number of photons emitted within the corresponding microfluidic detection chamber;
wherein each one of the photodiodes in the secondary array of photodiodes is covered to prevent ingress of light and wherein a secondary charge count derived from the secondary array of photodiodes is indicative of a dark count;
wherein an adjusted charge count is provided by using the dark count value to adjust the primary charge count.

In this way, it is possible to use the sensor to detect luminescence by detecting photon emissions. For example, such photon emissions may be released during a chemical reaction (chemiluminescence). By contrast with fluorescence, there is no need for any emission of light to excite the fluorescent material, and by contrast with absorbance, there is no need to emit light to be selectively absorbed/transmitted. Detecting photon emission from luminescence facilitates a larger dynamic scale than would be possible when detecting fluorescence or photon transmission following absorbance. Furthermore, there is no need for a light source or optics. This also eliminates any need for complex manufacturing and processing (such as may require additional components including those required for analogue to digital conversion or sigma delta modulation) and no need for calculations (e.g. derivative) and the result (plus the dark count rate) is obtained in real time because the results rely only on photon emission counting. All of this means the sensor lends itself well to low cost point of care diagnostic applications.

Furthermore, the sensor may be used to derive a result from a small volume of biomarker-containing liquid. One such biomarker-containing liquid may be a sample of blood containing a plasma. Potentially, the volume of blood may be sufficiently small as not to require a phlebotomy. No pre-analytical steps, such as centrifugation, are required. There is no requirement for a trained professional or standard laboratory equipment. The sensor can be portable and can be deployed in wide variety of settings, including in the home.

In another aspect of the disclosure, there is provided a method for sensing photons in liquids, the method comprising providing a sensor that comprises:
a microfluidic substrate, wherein the microfluidic substrate is non-transparent such as to prevent photons from passing through the microfluidic substrate, the microfluidic substrate comprising a planar face and a plurality of microfluidic detection chambers each configured to receive a volume of liquid of less than 5 micro litres, wherein each microfluidic detection chamber comprises a transparent aperture located in the planar face;
a semiconductor substrate comprising a plurality of single photon avalanche photodiodes facing the planar face of the microfluidic substrate and arranged in a plurality of primary arrays of photodiodes and at least one secondary array of photodiodes,
a plurality of sensing channels, wherein each sensing channel of the plurality of sensing channels comprises one of the microfluidic detection chambers and a corresponding one of the primary arrays of photodiodes arranged such that each photodiode in the primary array is aligned with and no more than 1,000 micrometres from its corresponding microfluidic detection chamber so as to receive photons emitted within the corresponding microfluidic detection chamber, wherein a primary charge count derived from the primary array of photodiodes provides a pre-adjusted value of photon count indicative of a total number of photons emitted within the corresponding microfluidic detection chamber;
wherein each one of the photodiodes in the secondary array of photodiodes is covered to prevent ingress of light and wherein a secondary charge count derived from the secondary array of photodiodes is indicative of a dark count;
wherein the method comprises:
triggering a luminescent reaction to occur in the microfluidic detection chambers;
obtaining, for each sensing channel, the primary charge count;
obtaining, for each sensing channel, the secondary charge count;
using the secondary charge count to adjust the primary charge count in order to calculate an adjusted charge count.

The disclosure also relates to a sensor card for use with a sensor card receiver comprising a microprocessor configured to receive and process data from the sensor card, wherein the sensor card comprises the microfluidic substrate.

As such, in another aspect of the disclosure there is provided a sensor card configured to be used with a sensor card receiver having a microprocessor so as to form the sensor as described, wherein the sensor card comprises:
a microfluidic substrate, wherein the microfluidic substrate is non-transparent such as to prevent photons from passing through the microfluidic substrate, the microfluidic substrate comprising a planar face and a plurality of microfluidic detection chambers each configured to receive a volume of liquid of less than 5 micro litres, wherein each microfluidic detection chamber comprises a transparent aperture located in the planar face; and
a plurality of sensing channels, wherein each sensing channel of the plurality of sensing channels comprises one of the microfluidic detection chambers.

In this way, non-fluid containing elements of the sensor may be provided in the sensor card receiver, separately from the microfluidic substrate. Accordingly, the sensor card, which receives blood products into the microfluidic substrate, may be disposable without needing its own microprocessor.

### FURTHER ASPECTS OF THE DISCLOSURE

Further aspects of the disclosure are set out in the following numbered clauses:
1. A microfluidic substrate (300, 500) comprising:
   a planar face;
   a blood inlet;
   a blood processing region configured to receive blood from the blood inlet and supply processed blood to a blood processing region outlet (530);
   a buffer supply circuit comprising a buffer inlet (550) configured to receive buffer and a buffer outlet (560);
   a first processing circuit (501) comprising:
   a first mixing region (570) configured to receive and facilitate mixing of processed blood from the blood processing region outlet (530) with buffer from the buffer outlet (560) such as to provide a blood-buffer mixture via a mixing region outlet (580);
   a first plurality of microfluidic detection chambers (710) each containing a first dry reaction reagent and configured to receive via the mixing region outlet (580) a volume of the blood-buffer mixture of less than 5 micro litres for dissolving the first dry reaction reagent, wherein the first dry reaction reagent is configured to trigger a luminescent reaction;
   wherein the microfluidic substrate (300, 500) is non-transparent such as to prevent or at least substantially restrict photons from passing through the microfluidic substrate (300, 500) from any one of the first plurality of microfluidic detection chambers (710) to any of the other first plurality of microfluidic detection chambers (710); and
   wherein each microfluidic detection chamber (710) of the first plurality of microfluidic detection chambers (710) comprises a transparent aperture located in the planar face.

In this way, it is possible to process a significantly smaller volume of blood than is conventionally required for many assays, including blood coagulation assays, and yet still to provide a highly accurate quantitative analysis by triggering a luminescent chemical reaction in the microfluidic detection chambers. Potentially, the volume of blood may be sufficiently small as not to require a phlebotomy. No pre-analytical steps, such as centrifugation, are required. It may therefore be possible to avoid need for a trained professional or standard laboratory equipment.
2. The microfluidic substrate (300, 500) of clause 1 wherein the blood processing region comprises a blood filter configured to release blood plasma, wherein the processed blood comprises the blood plasma.
3. The microfluidic substrate (300, 500) of clause 1 or clause 2 wherein the first blood processing circuit (501) comprises a first preliminary reagent chamber (590) downstream of the mixing region outlet (580) and upstream of the first plurality of microfluidic detection chambers (710) such as to facilitate absorbance of first preliminary reagent in the first preliminary reagent chamber (590) with the blood-buffer mixture in order to provide a first blood buffer preliminary reagent mix to the first plurality of microfluidic detection chambers (710).
4. The microfluidic substrate (300, 500) of any preceding clause wherein the buffer supply circuit further comprises a gas release chamber (555) between the buffer inlet (550) and the buffer outlet (560), wherein the gas release chamber (555) is configured to release gas bubbles from buffer received via the inlet (550) prior to output of the buffer to the buffer outlet (560).
5. The microfluidic substrate (300, 500) of any preceding clause wherein the blood processing region comprises a metering chamber between the blood inlet and the blood processing region outlet (530).
6. The microfluidic substrate of any preceding clause wherein the blood processing region further comprises a blood processing region flow control regulator (527) configured to regulate flow rate of processed blood at the blood processing region outlet (530) in order to influence dilution ratio of the blood-buffer mix.
7. The microfluidic substrate of any preceding clause wherein the buffer supply circuit further comprises a buffer control regulator (557) configured to regulate flow of buffer at the buffer outlet (560) in order to influence dilution ratio of the blood-buffer mix.
8. The microfluidic substrate of any preceding clause wherein the buffer supply circuit further comprises a buffer capsule, such as a blister capsule, containing buffer configured to be supplied to the buffer inlet (550).
9. The microfluidic substrate of clause 3 of any clause dependent upon clause 3 further comprising a first preliminary reagent chamber valve assembly (593) comprising the first preliminary reagent chamber (590), a bypass channel (591) that bypasses the first preliminary reagent chamber (590), and a confluence (592) downstream of the first preliminary reagent chamber (590) and the bypass channel (591), wherein the confluence is configured such that exit of first blood-buffer-preliminary reagent mix from the first preliminary reagent chamber (590) is triggered by arrival of processed blood from the bypass channel (591) so as to trigger opening of the first preliminary reagent chamber valve assembly.
10. The microfluidic substrate of any preceding clause wherein a ratio of buffer to processed blood at the mixing region (570) is determined by relative geometries of the blood processing region and the buffer supply circuit.
11. The microfluidic substrate of any preceding clause further comprising a second blood processing circuit (601) comprising:
   a second blood processing circuit mixing region (570) configured to receive and facilitate mixing of processed blood from the blood processing region outlet (530) with buffer from the buffer outlet (560) such as to provide diluted processed blood via a second blood processing circuit mixing region outlet (580);
   a second plurality of microfluidic detection chambers (720) each containing a second dry reaction reagent and configured to receive via the mixing region outlet (580) a volume of the blood-buffer mixture of less than 5 micro litres for dissolving the second dry reaction reagent;
   wherein the microfluidic substrate (300, 500) being non-transparent prevents or at least substantially restricts photons from passing through the microfluidic substrate (300, 500) from any one of the first or second plurality of microfluidic detection chambers (720) to any of the other first or second plurality of microfluidic detection chambers (720); and
   wherein each microfluidic detection chamber of the second plurality of microfluidic detection chambers (720) comprises a transparent aperture located in the planar face.
12. The microfluidic substrate of any preceding clause further comprising a planar layer over the planar face to retain liquid within each microfluidic detection chamber, wherein the planar layer is transparent at least in locations covering the transparent apertures in order to allow photons to pass though.
13. The microfluidic substrate of clause 12 wherein the planar layer provides a fluid contact angle of between 80 degrees and 100 degrees, preferably 90 degrees.
14. The microfluidic substrate of clause 12 or clause 13 wherein the planar layer comprises a planar foil.
15. The microfluidic substrate of any of clauses 12 to 14 wherein the planar layer comprises translucent or opaque regions in locations remote from the transparent apertures in order to restrict or prevent photon transmission in said locations.
16. The microfluidic substrate of clause 15 wherein the translucent or opaque regions comprise carbon doped planar layer.
17. The microfluidic substrate of any of clauses 12 to 16 wherein the planar layer comprises a matt surface.
18. The microfluidic substrate of any preceding clause further comprising an anti-fouling coating.
19. The microfluidic substrate of any preceding clause wherein the microfluidic substrate has an optical density attenuation coefficient of at least 100, preferably at least 1,000.
20. A sensor card comprising:
   the microfluidic substrate (300, 500) of any preceding clause; and
   a semiconductor substrate comprising a plurality of single photon avalanche photodiodes facing the planar face of the microfluidic substrate and arranged in a plurality of primary arrays of photodiodes and at least one secondary array of photodiodes, wherein each one of the plurality of primary arrays of photodiodes is arranged such that each photodiode in the primary array is aligned with and no more than 1,000 micrometres from a corresponding microfluidic detection chamber so as to receive photons emitted within the corresponding microfluidic detection chamber, and wherein each one of the photodiodes in the secondary array of photodiodes is covered to prevent ingress of light.
21. The sensor card of clause 20 wherein the semiconductor substrate comprises a planar surface and the planar surface of the printed circuit board is mounted parallel to the planar face of the semiconductor substrate.
22. The sensor card of clause 21 when dependent upon clause 12 or any clause dependent upon clause 12 wherein the semiconductor substrate is located on the planar layer.
23. The sensor card of any of clauses 20 to 22 wherein the semiconductor substrate and the microfluidic substrate comprise corresponding alignment features for assisting alignment between each microfluidic detection chamber and its corresponding primary array of photodiodes.
24. The sensor card of any of clauses 20 to 23 when dependent upon clause 11 or any clause dependent upon clause 11, wherein a first subset of the primary arrays of photodiodes corresponds with the first plurality of microfluidic detection chambers (710) and a second subset of the primary arrays of photodiodes corresponds with the second plurality of microfluidic detection chambers (720).
25. The sensor card of any of clauses 20 to 24 further comprising: a temperature sensor circuit comprising: a temperature sensor and a heat sink element for use in regulating a temperature of the material being sensed.
26. The sensor of any of clauses 20 to 25 wherein each one of the single photon avalanche photodiodes is configured to detect wavelengths within the visible spectrum.
27. The sensor of any of clauses 20 to 26 wherein each one of the single photon avalanche photodiodes has a response time of less than 10 nanoseconds, preferably less than 1 nanosecond, more preferably less than 100 picoseconds.
28. The sensor of any of clauses 20 to 27 wherein each one of the single photon avalanche photodiodes has a dynamic range of at least four orders of magnitude and preferably six orders of magnitude.
29. The sensor of any of clauses 20 to 28 wherein each one of the photodiodes in the secondary arrays of photodiodes is covered to prevent ingress of light by a metal layer applied directly to the semiconductor substrate.
30. The sensor of any of clauses 20 to 29 wherein the semiconductor substrate comprises an opaque coating across the face of the semiconductor substrate except in a region of the single avalanche photodiodes.
31. The sensor of any of clauses 20 to 30 further comprising a printed circuit board, wherein the semiconductor substrate is mounted to the printed circuit board.
32. The sensor of clause 31 when dependent upon clause 30 wherein the opaque coating covers the printed circuit board as well as the face of the semiconductor substrate except in a region of the single avalanche photodiodes.
33. A method for sensing photons in liquids, the method comprising using a microfluidic substrate (300, 500) in accordance with any of clauses 1 to 19, the method comprising:
   depositing a sample of blood on the blood filter (305);
   depositing a sample of buffer on the buffer inlet (550); and
   detecting photons emitted from each microfluidic detection chamber independently.
35. A method for sensing photons in liquids, the method comprising using a microfluidic substrate (300, 500) in accordance with clause 11 or any clause dependent upon clause 11, the method comprising:
   depositing a sample of blood on the blood filter (305);
   depositing a sample of buffer on the buffer inlet (550); and
   detecting photons emitted from each microfluidic detection chamber independently to determine a photon count for each microfluidic detection chamber.

### DRAWINGS

Examples of sensors in accordance with the present invention in accordance with the disclosure are now described with reference to the following drawings:

FIG. 1 shows a sensor assembly comprising a sensor in accordance with the disclosure;

FIG. 2 shows a sensor assembly comprising a sensor in accordance with the disclosure;

FIG. 3 shows a sensor card that forms a part of the sensor assembly shown in FIG. 2;

FIG. 4 shows the sensor card of FIG. 3 from an opposite side;

FIG. 5A shows a printed circuit board of the card of FIG. 3, including two integrated circuits with semiconductor substrates comprising the single photon avalanche photodiodes;

FIG. 5B shows a highly schematic enlarged view showing parts of the integrated circuits comprising the single photon avalanche photodiodes;

FIG. 6 shows a microfluidic substrate that forms a part of the sensor card of FIG. 3;

FIG. 7 shows a part of the microfluidic substrate of FIG. 6 alongside some elements of the integrated circuits of FIG. 5B;

FIG. 8 shows a cross sectional view of the sensor assembly of FIG. 2;

FIG. 9 shows parts of the sensor assembly of FIG. 2;

FIG. 10 shows a cross sectional view of the buffer capsule approaching a position at which it is actuated which may be effected by movement of the sensor card into the sensor card receiver in the direction of the arrow shown in FIG. 9;

FIG. 11 shows an embodiment of a microfluidic substrate (an alternative to that shown in FIG. 6) that forms a part of the sensor card of FIG. 3;

FIG. 12 shows a flow chart illustrating a high level process involved in conducing a blood analysis using a microfluidic substrate in accordance with the present disclosure; and

FIG. 13 is a highly schematic representation of the sensor assembly showing conceptually how the components interrelate.

### DETAILED DESCRIPTION

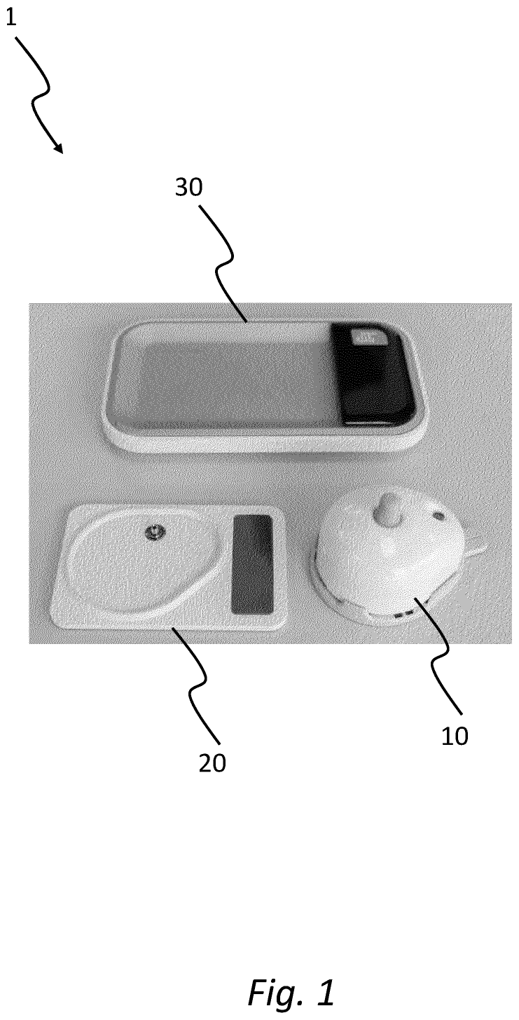

FIG. 1 shows a first embodiment of a sensor assembly 1 comprising a sensor card 20 including a sensor in accordance with the disclosure.

The sensor card 20 may comprise a plurality of parallel sensing channels, meaning a plurality of sensing channels that are processed in parallel (rather than necessarily being geometrically parallel).

The sensor assembly 1 comprises a sample collection device 10, the sensor card 20 and a sensor card receiver 30. The sample collection device 10 and the sensor card 20 may be single use. The sensor card receiver 30 may be multi-use.

The sample collection device 10 may be used to collect blood for testing. The sensor card 20 may be configured to receive the sample collection device 10 so as to receive from the sample collection device 10 the blood for testing.

As explained further below, the sensor card 20 may include microfluidic and electronic components.

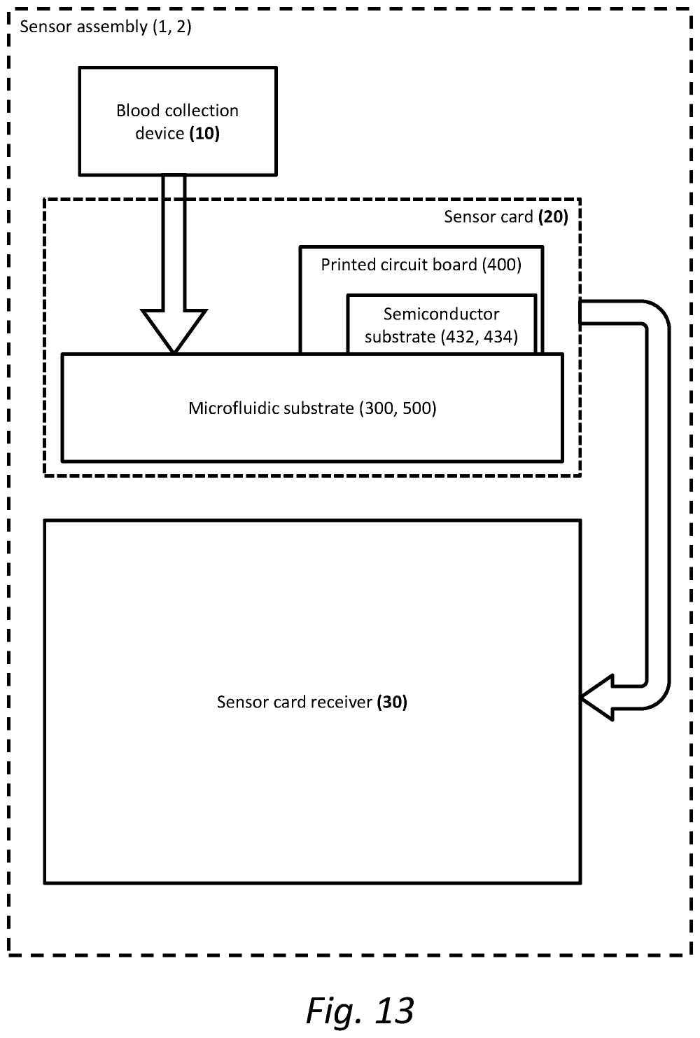

The interrelationship between the components of the sensor assembly 1 is shown in a highly schematic fashion in FIG. 13.

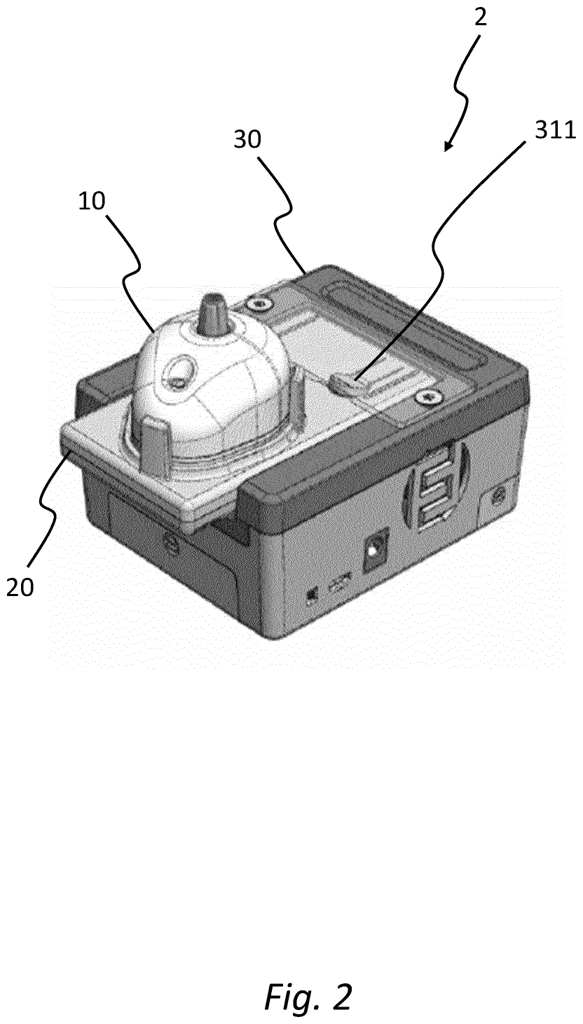

FIG. 2 shows a second embodiment of a sensor assembly 2 comprising a sensor card 20 including a sensor in accordance with the disclosure. The sensor assembly 2 comprises a sample collection device 10, a sensor card 20 and a sensor card receiver 30. The sensor card 20 comprises the sensor 20. The sample collection device 10 and the sensor card 20 may be single use. The sensor card receiver 30 may be multi-use. In FIG. 2, the components of the sensor assembly 2 are shown in mutual cooperation.

The conceptual interrelationship between the components of the second embodiment of the sensor assembly 2 is the same as that for the first embodiment of the sensor assembly 1 which is shown in a highly schematic form in FIG. 13.

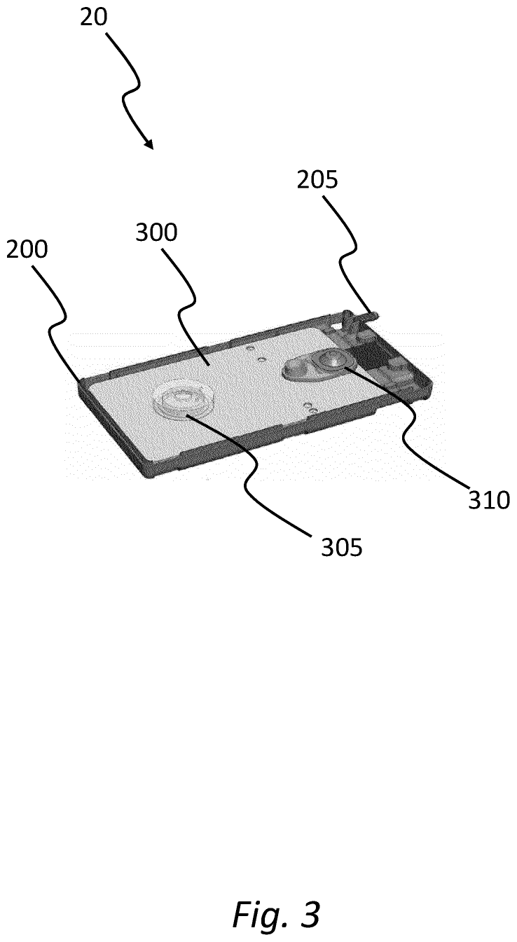

FIG. 3 shows parts of an embodiment of a sensor card 20 comprising a sensor in accordance with the present disclosure.

The sensor card 20 is configured to cooperate with a sample collection device 10 (not shown in FIG. 3) and with a sensor card receiver 30 (not shown in FIG. 3). In the view of FIG. 3, the upwardly facing surface of the sensor card 20 would face upwardly when interacting with the sensor card receiver 30 in the manner shown in FIG. 2.

The sensor card 20 comprises a housing 200 (only part of which is shown in FIG. 3) having an air inlet 205.

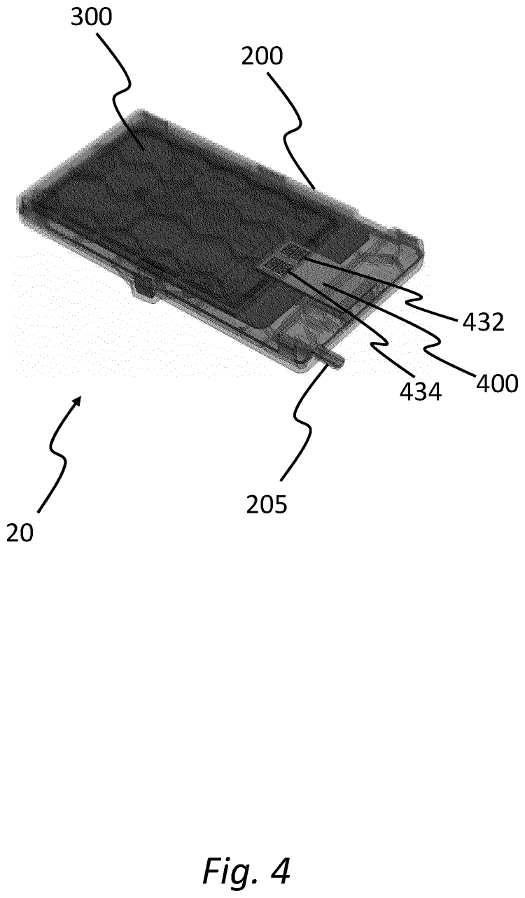

FIG. 4 shows the sensor card 20 from a different perspective to FIG. 3. In the view of FIG. 4, the upwardly facing surface of the sensor card would face downwardly when interacting with the sensor card receiver 30 in the manner shown in FIG. 2.

The sensor card 20 comprises a microfluidic substrate 300. The microfluidic substrate 300 may comprise a buffer capsule 310 (shown in FIG. 3) with a pierceable membrane by which buffer may be released from the buffer capsule 310 in order to dilute the sample and assist in carrying the sample through microfluidic channels of the microfluidic substrate 300. FIG. 3 also shows a filter assembly 303 of the microfluidic substrate 300. The filter assembly 303 may comprise a filter housing 304 and a filter 305. The filter 305 may be formed of a polysulfone material (PSM). The buffer capsule 310 may be actuated by a buffer capsule trigger switch 311, visible in FIG. 2.

Although the illustrated embodiments include a filter for separating blood plasma from the blood sample in order that blood plasma may be processed in the following stages, a blood filter is not essential. The sensor card 20 of the present disclosure may equally be deployed for unfiltered blood assays. Furthermore, the sensor card 20 of the present disclosure may alternatively be deployed for analysing samples other than blood.

Referring to FIG. 4, the sensor card 20 comprises the housing 200 (including air inlet 205) and contains the microfluidic substrate 300 and an electronic circuit board 400 mounted directly on the microfluidic substrate 300. The electronic circuit board 400 includes a pair of integrated circuits 432, 434, formed from semiconductor substrates 432, 434. In the illustrated example, the pair of integrated circuits 432, 434 is a pair of application-specific integrated circuits (ASICs) 432, 434.

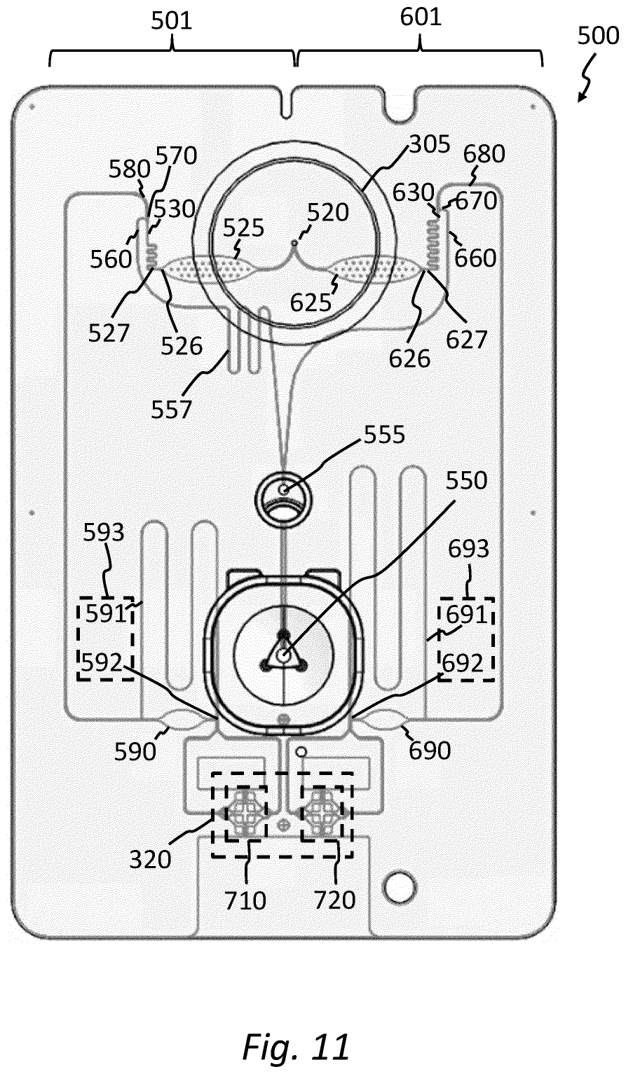

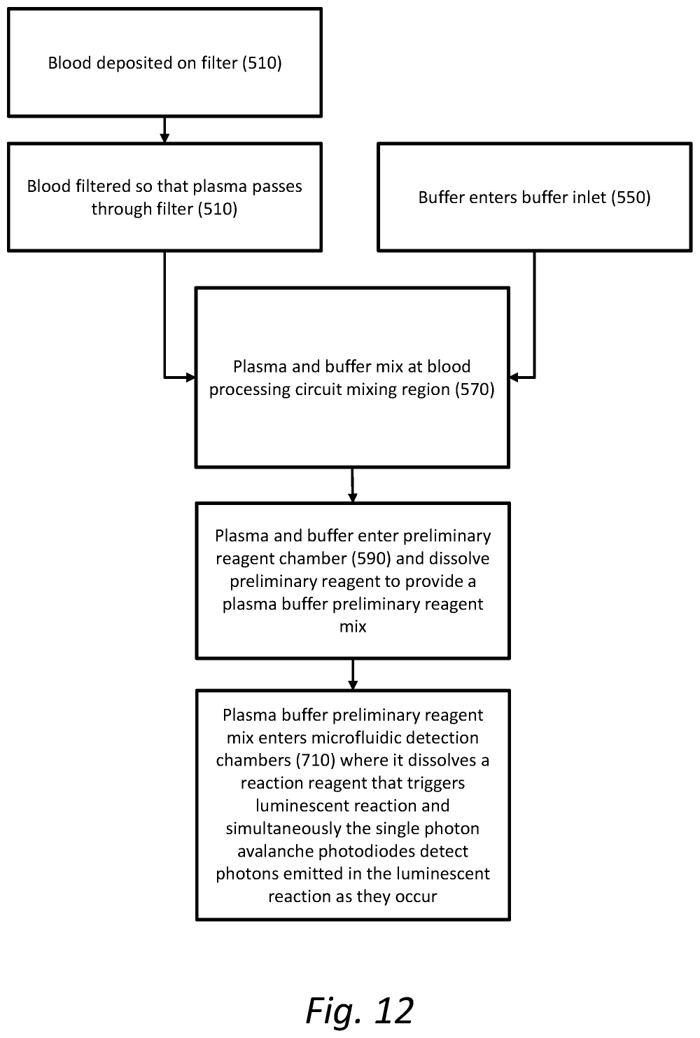

The microfluidic substrate 300 comprises multiple parallel microfluidic channels (not visible in FIG. 4 but more readily visible in the microfluidic substrate 500 shown in FIG. 11), each of which parallel multiple microfluidic channels may correspond to a separate sensing channel. In the illustrated embodiments there are 16 parallel microfluidic channels, as will be explained further below. In some embodiments, a first subset of the microfluidic channels may be used to test for one or more specific markers and a second subset of the microfluidic channels may be used to test for another set of markers.

Fluids are drawn through the appropriate channels of the microfluidic substrate 300 by capillary action.

The microfluidic substrate 300 comprises a planar surface containing an aperture for each microfluidic detection chamber. In this way, on assembly of the microfluidic substrate 300 with the electronic circuit board 400, planar surfaces of the semiconductor substrates 432, 434 face the planar surface of the microfluidic substrate 300 such that the single photon avalanche photodiodes are close to and aligned with the appropriate microfluidic detection chamber of the plurality of microfluidic detection chambers in the microfluidic detection chamber area 320.

The microfluidic substrate 300 is opaque to prevent cross-talk between sensing channels. Specifically, at least that part of the microfluidic substrate 300 that contains the microfluidic detection chambers is opaque. In FIG. 4, this is the part of the microfluidic substrate 300 that is immediately adjacent (beneath) the pair of integrated circuits 432, 434. In one embodiment, part or all of the microfluidic substrate 300 may be of (e.g. black) cyclic olefin copolymer (COC), which may be opaque.

That the microfluidic substrate is opaque may be alternatively be described as the microfluidic substrate being non-transparent. The microfluidic substrate being opaque may mean that the microfluidic substrate has an optical density attenuation coefficient of at least 100, preferably at least 1,000.

In addition, to reduce further a risk of light escaping from a particular microfluidic detection chamber where it is produced, the surfaces of the microfluidic substrate 300 may be matt rather than glossy.

The microfluidic substrate may be covered with a planar layer to retain liquid within each microfluidic detection chamber whilst also allowing photons to escape from each microfluidic detection chamber to its corresponding array of single photon avalanche photodiodes, as discussed further below. The planar layer may be entirely transparent or may be transparent only in those regions adjacent to each microfluidic detection chamber so as to allow transmission through the planar layer to the corresponding array of single photon avalanche photodiodes. The planar layer may comprise a foil. In an alternative arrangement, the planar layer may comprise a plastic.

FIG. 11 shows one embodiment of the microfluidic substrate 500 in more detail. The microfluidic substrate 500 comprises a planar face. The microfluidic substrate further comprises a blood inlet (not separately shown in FIG. 11) and a blood filter 305 configured to receive blood from the blood inlet and to release blood plasma via a blood filter outlet 520.

The microfluidic substrate 500 further comprises a blood processing region. The blood plasma processing region is configured to receive blood (e.g. via the blood collection device 10 shown in FIGS. 1 and 2) and to process that blood before outputting that processed blood via a blood processing region outlet 530.

In the microfluidic substrates 500 of FIG. 11, the blood processing region is specifically a blood plasma processing region. (However, a blood processing region which processes whole unfiltered blood falls within the scope of the disclosure.) The blood plasma processing region comprises a filter 305 and a blood filter outlet 520. The filter 305 is configured to filter incoming blood and to release blood plasma to the blood filter outlet 520 and onwards to the blood processing region outlet 530.

The microfluidic substrate 500 further comprises a buffer supply circuit comprising a buffer inlet 550 configured to receive buffer and a buffer outlet 560. The microfluidic substrate 500 further comprises a buffer capsule 310 (not shown in FIG. 11) configured to supply buffer to the buffer inlet 550.

The microfluidic substrate 500 further comprises a first blood processing circuit 501 and a second blood processing circuit 601. The second blood processing circuit 601 operates in parallel with the first blood processing circuit 501.

The first blood processing circuit 501 comprises a first mixing region 570 configured to receive and facilitate mixing of processed blood (e.g. plasma) from the blood processing region outlet 530 with buffer from the buffer outlet 560 such as to provide a blood-buffer mixture (specifically, a mixture of blood plasma with buffer) via a mixing region outlet.

The blood processing region outlet 530 may have a smaller cross section than the buffer outlet 560. The cross section of the blood processing region outlet 530 may be such that it acts as a passive stop valve wherein processed blood does not pass into the first mixing region 570 until buffer arrives at the buffer outlet 560 so as to draw the processed blood through the blood processing region outlet 530 by capillary action.

The first blood processing circuit 501 further comprises a first plurality of microfluidic detection chambers 710 each containing a first dry reaction reagent. Each of the first plurality of microfluidic detection chambers 710 is configured to receive via the first blood processing circuit mixing region outlet 580 a volume of the blood (plasma)-buffer mixture of less than 5 micro litres for dissolving the first dry reaction reagent, wherein the first dry reaction reagent is configured to trigger a luminescent reaction.

The microfluidic substrate 500 is non-transparent such as to prevent or at least substantially restrict photons from passing through the microfluidic substrate 500 from any one of the first plurality of microfluidic detection chambers 710 to any of the other first plurality of microfluidic detection chambers 710.

Each microfluidic detection chamber 710 of the first plurality of microfluidic detection chambers 710 comprises a transparent aperture located in the planar face.

The first blood processing circuit 501 comprises a first preliminary reagent chamber 590 downstream of the mixing region outlet 580 and upstream of the first plurality of microfluidic detection chambers 710 such as to facilitate absorbance of the first preliminary reagent in the first preliminary reagent chamber 590 with the blood (plasma)-buffer mixture in order to provide a first buffer preliminary reagent mix to the first plurality of microfluidic detection chambers 710.

The buffer supply circuit further comprises a gas release chamber 555 between the buffer inlet 550 and the buffer outlet 560. The gas release chamber 555 is configured to release gas bubbles from buffer received via the inlet 550 prior to output of the buffer to the buffer outlet 560. The gas release chamber 555 may comprise a gas barrier which gas bubbles are unable to penetrate.

The blood processing region comprises a metering chamber between the blood filter outlet 520 and the blood processing region outlet 530.

The blood processing region further comprises a flow control regulator 527 configured to regulate the flow rate of the blood plasma so as to influence the dilution ratio of the blood plasma with the buffer.

Similarly, the buffer supply circuit further comprises a buffer control regulator 557 so as to influence the dilution ratio of the blood plasma with the buffer.

The buffer supply circuit further comprises a buffer capsule containing buffer configured to be supplied to the buffer inlet 550.

The microfluidic substrate further comprises a first preliminary reagent chamber valve assembly 593. The first preliminary reagent chamber valve assembly 593 comprises the first preliminary reagent chamber 590, a bypass channel 591 that bypasses the first preliminary reagent chamber 590, and a confluence 592 downstream of the first preliminary reagent chamber 590 and the bypass channel 591.

The first preliminary reagent chamber valve assembly 593 receives a blood (plasma)-buffer mixture from the first blood processing circuit mixing region outlet and some of that blood (plasma)-buffer mixture travels into the reagent chamber 590 while the remainder of that blood (plasma)-buffer mixture travels into the bypass channel 591. It may be that blood (plasma)-buffer mixture only travels into the bypass channel 591 once the reagent chamber 590 is full. A length of the bypass channel may be selected to delay arrival of the blood (plasma)-buffer mixture at the confluence 592. The confluence may be configured such that content of the first preliminary reagent chamber 590 cannot be drawn through the confluence 592 until blood (plasma)-buffer mixture arrives via the bypass channel at the confluence 592, whereby it draws the content of the first preliminary reagent chamber 590 via a capillary action. In this way, the first preliminary reagent chamber valve assembly 593 only opens to allow content of the first preliminary reagent chamber 590 to arrive at the first plurality of microfluidic detection chambers 710 after a delay period during which time it is expected that the first preliminary reagent has been dissolved into the blood (plasma)-buffer mixture in the first preliminary reagent chamber 590.

A ratio of buffer to blood plasma at the mixing region 570 is determined by relative geometries of the blood processing region and the buffer supply circuit.

The second blood processing circuit 601 comprises a second blood processing circuit mixing region 670 configured to receive and facilitate mixing of processed blood (e.g. plasma) from the blood processing region outlet 630 with buffer from the buffer outlet 660.

In this way, blood-buffer mixture (specifically, a mixture of blood plasma with buffer) is provided via a second blood processing circuit mixing region outlet 680.

A ratio of the blood-buffer mixture provided from the second blood processing circuit mixing region outlet 680 may be different from a ratio of the blood-buffer mixture provided from the first blood processing circuit mixing region outlet 580.

The second blood processing circuit 601 may have features that correspond with those of the first blood processing circuit 501. In FIG. 11, where the second blood processing circuit 601 has a feature having a corresponding feature of the first blood processing circuit 501, the reference numeral is incremented by 100 (so, for example, the buffer outlet 560 of the first blood processing circuit 501 corresponds with the buffer outlet 660 of the second blood processing circuit 601).

The second blood processing circuit 601 comprises a second plurality of microfluidic detection chambers 720 each containing a second dry reaction reagent and configured to receive via the mixing region outlet 680 a volume of the diluted blood plasma of less than 5 micro litres for dissolving the second dry reaction reagent.

The second blood processing circuit 601 further comprises a second preliminary reagent chamber 690 downstream of the mixing region outlet 680 and upstream of the first plurality of microfluidic detection chambers 720 such as to facilitate absorbance of second preliminary reagent in the second preliminary reagent chamber 690 with the diluted blood plasma in order to provide a second plasma buffer preliminary reagent mix to the second plurality of microfluidic detection chambers 720.

The second preliminary reagent may be different from the first preliminary reagent in order to effect a different reaction in the second blood processing circuit 601 from in the first blood processing circuit 501. For example, the first preliminary reagent may be targeting a specific analyte while the second preliminary reagent may be for a global assay, or vice versa.

The non-transparency of the microfluidic substrate 500 prevents or at least substantially restricts photons from passing through the microfluidic substrate 500 from any one of the first or second plurality of microfluidic detection chambers 720 to any of the other first or second plurality of microfluidic detection chambers.

Each microfluidic detection chamber of the second plurality of microfluidic detection chambers 720 comprises a transparent aperture located in the planar face.

A planar foil is provided over the planar face to retain liquid within the various microfluidic channels of the microfluidic substrate 500, including within each microfluidic detection chamber 710, 720. The planar foil is transparent at least in locations covering the transparent apertures in order to allow photons to pass though. Alternatives to foil are possible, as long as they (a) retain liquid within the channels and (b) allow photons to pass out of each microfluidic detection chamber 710, 720 to its respective array of photodiodes.

The planar foil may comprise translucent or opaque regions in locations remote from the transparent apertures in order to restrict or prevent photon transmission in said locations.

The translucent or opaque regions may comprise carbon doped planar foil. The planar foil may comprise a matt surface.

The microfluidic substrate 500 may comprise an anti-fouling coating.

The microfluidic substrate 500 may have an optical density attenuation coefficient of at least 100, preferably at least 1,000.

When the sensor card is assembled by bringing together the microfluidic substrate 500 with the semiconductor substrate(s), the semiconductor substrate may be located on the planar foil.

The semiconductor substrate and the microfluidic substrate may comprise corresponding alignment features for assisting alignment between each microfluidic detection chamber and its corresponding primary array of photodiodes.

A first subset of the primary arrays of photodiodes corresponds with the first plurality of microfluidic detection chambers 710 and a second subset of the primary arrays of photodiodes corresponds with the second plurality of microfluidic detection chambers 720.

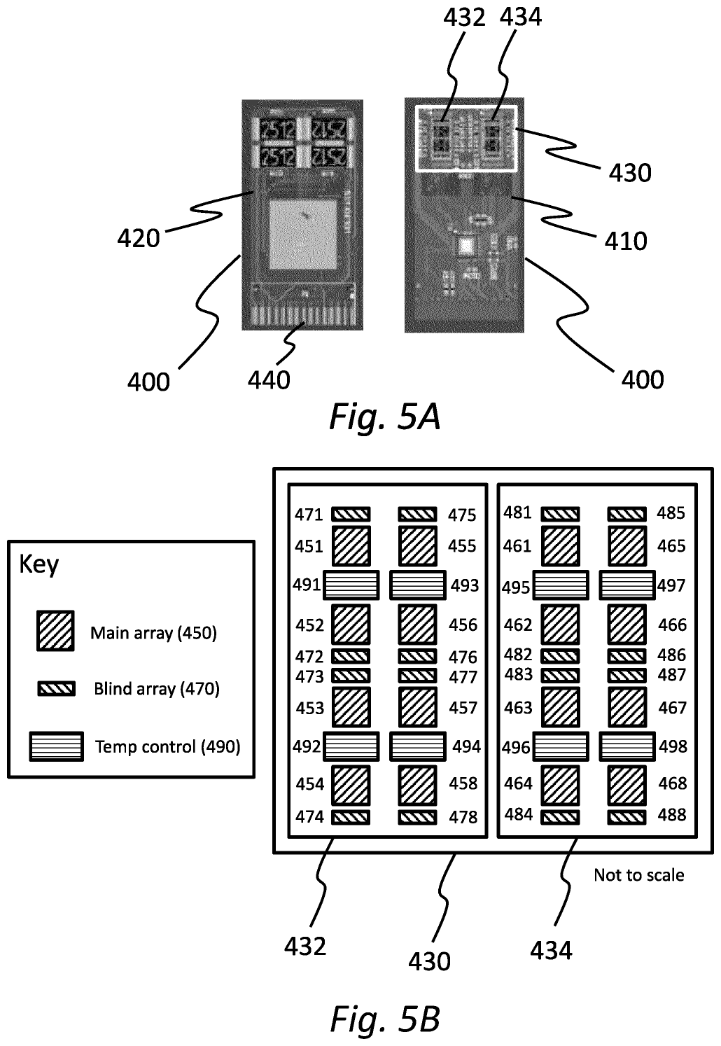

The electronic circuit board 400, shown in more detail in FIG. 5A, comprises a printed circuit board 400 having a first face 410 and a second face 420. The first face 410 is configured to face inwardly towards the microfluidic substrate 300 and the second face 420 is configured to face outwardly away from the microfluidic substrate 300. The second face 420 comprises contacts 440 configured to correspond with corresponding contacts (not shown) on the sensor card receiver 30. The first face 410 comprises a photodiode zone 430.

As seen in FIGS. 5A and 5B, the photodiode zone 430 comprises a first application-specific integrated circuit (ASICs) 432 and a second application-specific integrated circuit 434, each comprising a plurality of photodiodes. The photodiodes are single photon avalanche photodiodes. Each single photon avalanche photodiode may have a dynamic range of at least four orders of magnitude, and more preferably six orders of magnitude.

In accordance with regular single photon avalanche photodiode operation, counting individual photons involves counting the number of output charge pulses within a measurement period.

By providing the single photon avalanche photodiodes on few (in the illustrated embodiment two) application-specific integrated circuits 432, 434, planarity of photodiode arrays may be maximised meaning that when the application-specific integrated circuits 432, 434 are assembled adjacent the microfluidic detection chambers, scope for ingress of light is minimised.

The single photon avalanche photodiodes may each be configured to detect a broad range of wavelengths. This means they are able to detect luciferase photon emission, which tends to produce a broad-spectrum emission in the visible spectrum. In a specific implementation, each single photon avalanche photodiode has a quantum efficiency of at least 30% for wavelengths of between 400 and 650 nm and a quantum efficiency of at least 50% for wavelengths of between 450 and 500 nm. The single photon avalanche photodiodes may each be configured to have a response time of less than 10 nanoseconds, or preferably less than 1 nanosecond or more preferably less than 100 picoseconds. The single photon avalanche photodiodes may each have a dynamic range of at least four orders of magnitude, and more preferably six orders of magnitude.

In the specific embodiment illustrated in FIG. 5B, the photodiode zone 430 comprises 16 primary arrays 450 of photodiodes (451, 452, 453, 454, 455, 456, 457, 458, 461, 462, 463, 464, 465, 466, 467, 468), one for each of the 16 microfluidic channels. Each one of the primary arrays 450 may comprise 64 single photon avalanche photodiodes arranged in an 8×8 grid. The primary arrays 450 may also be referred to as main arrays 450.

The photodiode zone 430 further comprises 16 secondary arrays 470 of photodiodes (471, 472, 473, 474, 475, 476, 477, 478, 481, 482, 483, 484, 485, 486, 487, 488), one for each of the 16 primary arrays 450. Each secondary array 470 may comprise 16 single photon avalanche photodiodes arranged in a 8×2 grid. Each photodiode in each of the secondary arrays 470 is covered to prevent ingress of light. The secondary arrays 470 may also be referred to as blind arrays 470.

Although in the illustrated embodiment of FIG. 5B, there are 16 secondary arrays 470 of photodiodes, with a one-to-one correspondence with the 16 primary arrays 450 of photodiodes, this is not essential. It may be that each application-specific integrated circuit 432, 434, has only a single secondary array 470 to provide a dark count for all primary arrays 450 on the application-specific integrated circuit 432, 434. Alternatively, there may be more than one secondary array 470 per application-specific integrated circuit 432, 434 but still fewer than the total number of primary arrays 450 on the application-specific integrated circuit 432, 434.

In the illustrated embodiment of FIG. 5B, it can be seen that the area of each secondary array 470 is different to the area of each primary array 450. The different areas may give rise to different counts and to different edge effects. Such differences may need to be compensated for when providing the dark count.

The photodiode zone may further comprise 8 temperature sensor circuits 490 (each individually numbered as 491, 492, 493, 494, 495, 496, 497, 498). Thus, there is one temperature sensor circuit 490 per pair of primary arrays 450 of photodiodes.

Alternatively, the photodiode zone may comprise 4 temperature sensor circuits (not illustrated). In one example, the 4 temperature sensor circuits may occupy the same or a similar area to the temperature sensor circuits shown in FIG. 5B. In a further alternative, the photodiode zone may comprise fewer than 4 temperature sensor circuits (not illustrated).

In a further alternative, the temperature sensor circuits 490 may be substituted for an additional dark count single photon avalanche photodiode array in combination with a calibration mechanism for equating dark count to temperature.

In the illustrated embodiment, each of the 16 primary arrays 450 of photodiodes corresponds with and is adjacent to one of the 16 secondary arrays 470 of photodiodes.

The photodiode zone 430 has a geometrical arrangement which is such that a distance between each primary array and its corresponding secondary array is the same as a distance between each other primary array 450 and its corresponding secondary array 470.

Preferably, the single photon avalanche photodiodes of the primary arrays 450 have the same specification as the single photon avalanche photodiodes of the secondary arrays 470.

Each of the 16 secondary arrays 470 of photodiodes is covered with an opaque covering intended to prevent ingress of light or to reduce it to a level where it is negligible in terms of the individual number of photons that might be expected to penetrate the opaque covering. In preferred embodiments, the opaque covering of the secondary arrays is provided by a metal layer on the semiconductor substrate. (Other options for providing the opaque covering fall within the scope of the disclosure.) The metal layer may be embedded as part of the semiconductor substrate manufacture. The metal layer may be a metal coating. However it is formed, the opaque covering entirely covers each and every one of photodiodes in each of the secondary arrays.

In this way, a dark count may be obtained from each secondary array. A dark count is indicative of charge generated in the secondary array, notwithstanding that the secondary array is covered and therefore would not generally be expected to receive photons. Such charge may be generated as a result of imperfections in the photodiode, and such charge may vary with temperature. In any case, the secondary charge count may be indicative of a charge generated in the secondary array without being the result of photon emission in the microfluidic detection chamber. In the case of the illustrated embodiment, where each secondary array 470 of photodiodes comprises 16 photodiodes and each primary array 450 of photodiodes comprises 64 photodiodes, the secondary count obtained from each secondary array 470 may be indicative of one quarter (=16/64) of that arriving at the corresponding primary array 450 without having been released from its corresponding microfluidic detection chamber. In some arrangements, the comparison may in fact be between a median signal for the (e.g. 64) photodiodes in the primary array and a median signal for the (e.g. 16) photodiodes in the secondary array.

The eight temperature control circuits may be arranged such that each temperature control circuit is equidistant between each of two of a pair of adjacent primary arrays.

In this way, while each secondary array has one corresponding primary array, each temperature control circuit has two corresponding primary arrays.

The temperature sensor circuits 490 may comprise one or more sensors (not shown) for sensing temperature in the vicinity of the temperature sensor circuit 490. The temperature sensor circuits 490 may comprise one or more heating elements (not shown) and one or more heat sinks (not shown). The temperature sensor circuits 490 may also be located adjacent a thermal conduit (not shown) by which air flow may reach the temperature sensor circuits 490.

In the illustrated embodiment of FIG. 5B, a first half of the 16 primary arrays (for example, those with reference numerals 451, 452, 453, 454, 455, 456, 457, 458), a first half of the 16 secondary arrays (for example, those with reference numerals 471, 472, 473, 474, 475, 476, 477, 478) and a first half of the 8 temperature control circuits (for example, those with reference numerals 491, 492, 493, 494) are located on the first integrated circuit 432. A second half of the 16 primary arrays (for example, those with reference numerals 461, 462, 463, 464, 465, 466, 467, 468), a second half of the 16 secondary arrays (for example, those with reference numerals 481, 482, 483, 484, 485, 486, 487, 488), and a second half of the 8 temperature control circuits (for example, those with reference numerals 495, 496, 497, 498) are located on the second integrated circuit 434.

The electronic circuit board 400 may be partially over-moulded to cover elements including bonding wires which may protrude from the face of the electronic circuit board 400.

In a preferred embodiment, each primary array may have an area of the order of 500 μm×500 μm, or of the order of 450 μm×450 μm, or 480 μm×480 μm. In a preferred embodiment, a distance between a centre of each primary array and each adjacent primary array may be between 1,100 μm and 1,150 μm. In a preferred embodiment, a distance between each primary array and its corresponding secondary array may be between 175 μm and 290 μm.

Each single photon avalanche photodiode in either the primary array 450 or the secondary array 470 may, for example, have an area of approximately 62.5 μm×62.5 μm. In a further example, each single photon avalanche photodiode in either the primary array 450 or the secondary array 470 may have an area of approximately 50 μm×50 μm.

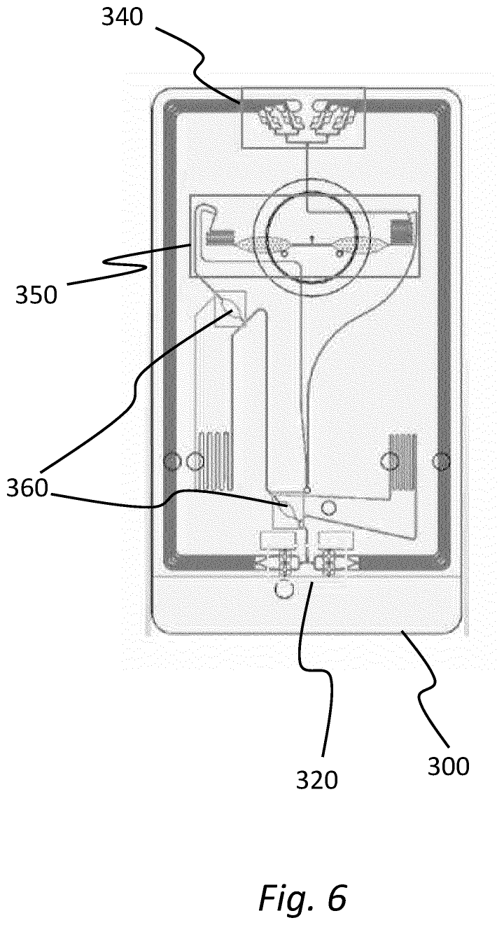

FIG. 6 shows an alternative embodiment of a microfluidic substrate 300 to that shown in FIG. 11. The microfluidic substrate 300 comprises a plurality of microfluidic channels by which the sample may travel through the microfluidic substrate 300. The microfluidic substrate further comprises a dilution zone 350 configured to dilute the sample and to separate the sample into multiple channels, a spotting chamber area 340 comprising a plurality of spotting chambers and trigger lines, downstream spotting chambers 360 and a microfluidic detection chamber area 320.

The sample may be supplied to the microfluidic substrate 300, 500 from the sample collection device 10. (Note that a range of sample collection devices may be appropriate, and the sample collection device 10 shown in the Figures is merely one example of many.) As mentioned, the microfluidic substrate 300 may comprise a filter assembly 303 that may comprise a filter housing 304 and a filter 305. In the case of a blood sample, the filter 305 may act to separate blood cells from plasma. In the case of a blood coagulation assay, the blood may be citrated blood. The sample collection device 10 may be a capillary blood collection device for use by a patient, or may be a blood tube with whole blood collected by phlebotomy by trained personnel.

The microfluidic substrate 300, 500 may be coated with an antifouling, hydrophilic coating to improve wettability and thereby assist in the passage of small volumes of liquid around the microfluidic channels which may have a cross sectional widths of tens to hundreds of microns (μm). In some embodiments, the hydrophilicity of the coating may be selected in order to result in a static contact angle of the sample in question of between 53° and 68° or approximately 56°, and so as to result in an advancing contact angle of between 65° and 85°, where the advancing contact angle is the contact angle at the advancing front of the diluted blood plasma.

Functional reagents may be used in some or all of the channels to quantify disease-specific parameters. These reagents may be applied to spotting chambers and/or detection chambers of the microfluidic substrate 300, 500 during functionalisation of the microfluidic substrate 300, 500. The reagents may be spotted into the necessary chamber(s) and air dried. After drying, the microfluidic substrate 300, 500 is laminated with a transparent foil and a blister is applied. The foil may have an anti-fouling capacity and may have a contact angle of 90 degrees. A filter (which serves as a plasma separation membrane) may then be applied using adhesive and covered with a cap.

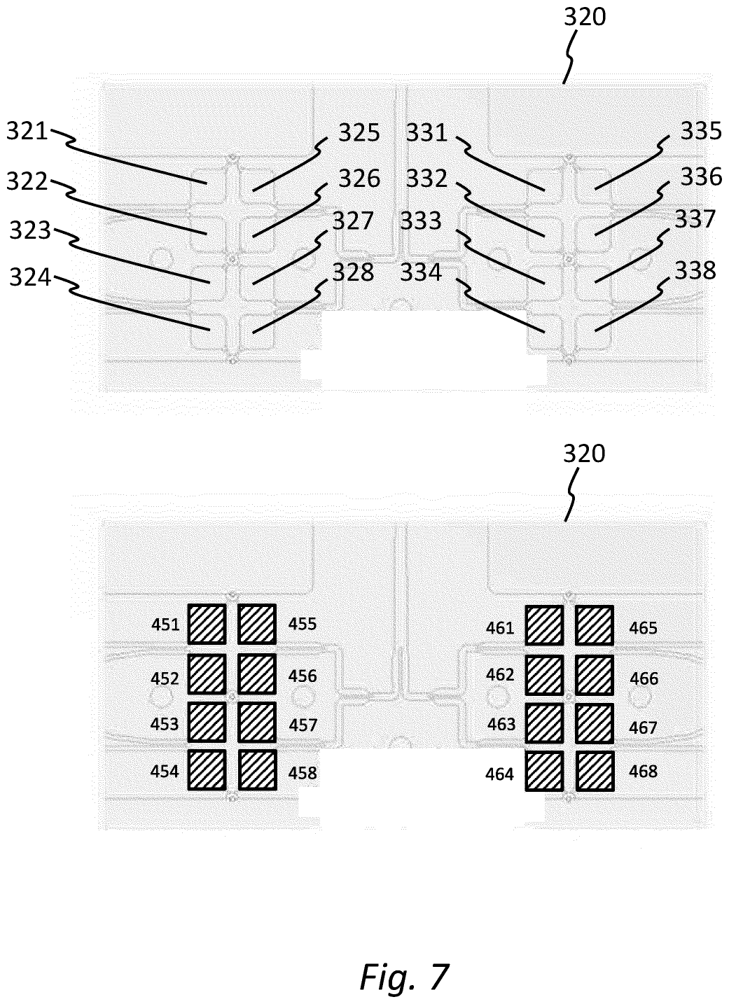

FIG. 7 shows the microfluidic detection chamber area 320, comprising 16 microfluidic detection chambers (321, 322, 323, 324, 325, 326, 327, 328, 331, 332, 333, 334, 335, 336, 337, 338). In this embodiment, each of the 16 microfluidic detection chambers has dimensions of 800 μm×800 μm×180 μm, meaning that each microfluidic detection chamber has a volume of 115 nL. While the illustrated embodiment has microfluidic detection chambers each having a volume of 115 nL, the disclosure covers small volume test chambers of up to 5 micro litre. The 800 μm×800 μm area of each microfluidic detection chamber aligns with the area of its corresponding single photon avalanche photodiode (e.g. 480 μm×480 μm). The depth of the microfluidic detection chamber (e.g. 180 μm) may be selected at least in part for fluid flow behaviour into the microfluidic detection chamber. As explained elsewhere, the liquid that reaches the microfluidic detection chamber may be diluted relative to the liquid (blood) first received into the sensor card. Also, the liquid may be filtered then diluted. For example, blood may be filtered to analyse only plasma and the plasma may be diluted before it reaches the microfluidic detection chamber.

FIG. 7 also shows, in highly schematic form, how the 16 primary arrays 450 of photodiodes (451, 452, 453, 454, 455, 456, 457, 458, 451, 452, 453, 454, 455, 456, 457, 458) of the photodiode zone 430 align with the 16 microfluidic detection chambers (321 322, 323, 324, 325, 326, 327, 328, 329, 330, 331, 332, 333, 334, 335, 336). Note that FIG. 7 shows the 16 primary arrays in isolation from the remaining features of the photodiode zone 430 shown in more detail in FIG. 5B.

Alignment features may be provided such that when the electronic circuit board 400 is fastened to the non-transparent microfluidic substrate 300, 500 during manufacture, alignment of each primary array of photodiodes to its corresponding microfluidic detection chamber can be optimised within a specific degree of tolerance so as to minimise the possibility that any light emitted within the microfluidic detection chamber in question will reach anywhere other than the corresponding primary array of photodiodes. The electronic circuit board 400 may be positioned on the microfluidic substrate 300, 500 by a pick and place robot.

Fastening of the electronic circuit board 400 to the microfluidic substrate 300, 500 during manufacture may involve use of an intermediate foil of low thickness, for example less than 50 μm, for example 25 μm, or 36 μm, or 40 μm. Adhesive may be used to fasten the foil in situ. The adhesive may have a thickness of 15 μm. The foil may be configured to maximise transparency in a direction orthogonal to the plane of the foil whilst minimising transparency in a direction parallel to the plane of the foil. In this way, transfer of light from the microfluidic detection chamber to its corresponding primary array of photodiodes may be maximised, whilst so-called cross talk of light from one microfluidic detection chamber to any other photodiode array (whether primary or secondary) may be minimised.

Cross-talk may be reduced by reducing the thickness of the transparent foil. Alternatively, or in addition, the transparency of the foil may be reduced, optionally in specific locations. For example, in some implementations, the foil may be doped with carbon between arrays of single photon avalanche photodiodes in order to minimise transfer of photons laterally between arrays. Other techniques may be employed in order to damp cross-talk. Surfaces of the microfluidic substrate 300, 500 may be finished with a matt rather than a glossy surface.

Once the electronic circuit board 400 has been fastened to the microfluidic substrate 300, 500, the combination of the two may be incorporated within a housing or envelope.

The resulting assembly of microfluidic substrate 300, 500 with electronic circuit board 400 may be such that a distance between each microfluidic detection chamber and its corresponding primary array may be no more than 1,000 μm or preferably no more than 200 μm and in some embodiments may be no more than 100 μm. The distance may be that measured along a line perpendicular a photon-receiving surface comprising the array of single photon avalanche photodiodes, starting at the centre of the photon-receiving surface and finishing at the closest boundary of its corresponding microfluidic detection chamber (that being the top of the microfluidic detection chamber, in the orientation of use).

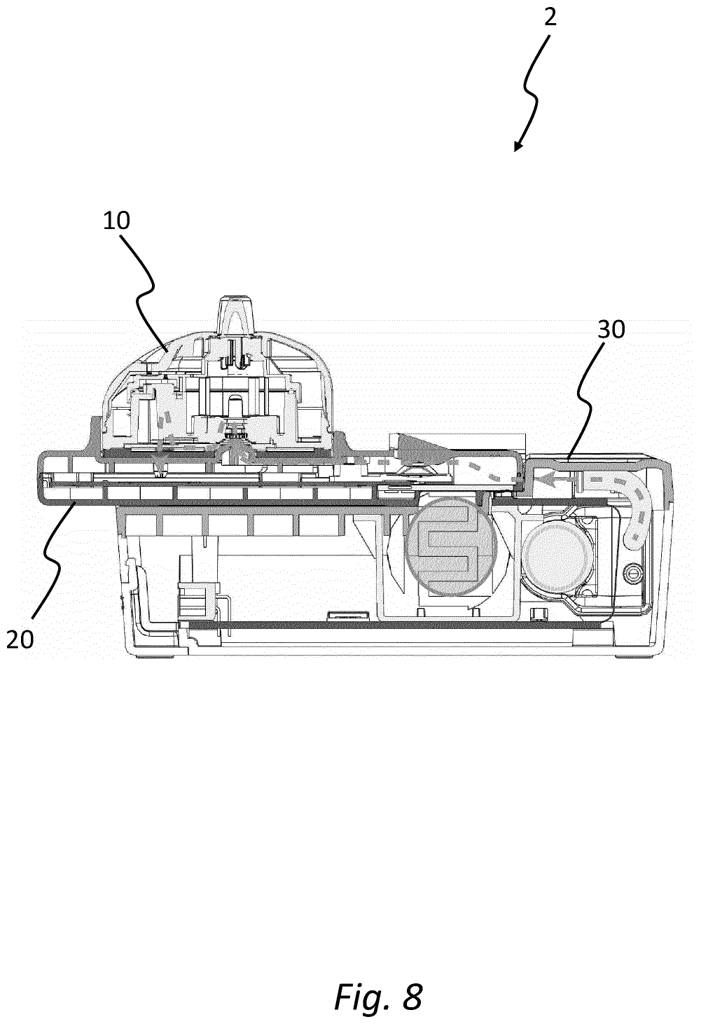

FIG. 8 shows a cross sectional view of the sensor assembly 1 comprising the sample collection device 10, the sensor card 20 and the sensor card receiver 30.

The sensor card receiver 30 comprises a processor (microprocessor) that cooperates with the electronic circuit board 400 of the sensor card 20. The sensor card receiver 30 may also comprise wireless communication technology to facilitate communication between the sensor card receiver 30 and an external device. For example, the sensor card receiver 30 may cooperate with a software application that may be installed on another device, such as a mobile device (e.g. phone, tablet) of a user, or a remote server, in order to send and receive data. Such data may include user identification data for linking with the sample. Such data may also include result data transmitted from the processor to the mobile device for the information of the user, or transmitted from the processor to a server, for example to be stored on a database.

Processing of the pre-adjusted value for each detection chamber and the dark count value associated with that detection chamber (in order to obtain the adjusted value) may be carried out on the electronic circuit board 400.

The sensor card receiver 30 may comprise a fan (not shown) for use in conjunction with the temperature sensor circuits 490 and may comprise a pump (not shown) for effecting the release of blood from the sample collection device 10. The processor may be configured to control operation of the pump to facilitate flow of blood from the sample collection device 10 into the sensor card 20. The processor may also be configured to receive data from the temperature sensor circuits 490 in order to determine an extent to which the measured temperature sensed by the temperature control circuits deviates from a target temperature. On the basis of that analysis, the processor may decide circumstances in which a temperature needs to be increased or reduced, and may be configured to send instructions to facilitate changes in temperature. In an event that the processor determines that a temperature is to be increased, the processor may send instructions to the heating elements to effect an increase in temperature in the sensor card 20. The heating elements may comprise one or more resistors mounted on the electronic circuit board 400 adjacent to the application-specific integrated circuits (ASICs) 432, 434. In an event that the processor determines that a temperature is to be reduced, the processor may be configured to control operation of the fan to facilitate flow of air in proximity to the heat sinks to effect a reduction in temperature in the sensor card 20.

In the context of a blood coagulation assay, it may be important to ensure that the temperature of the sample is maintained as close as possible to 37° C. As such, the processor may be configured to deploy the heating elements when the measured temperature drops to, for example, 36.9° C., or 36.8° C., or 36.7° C., or 36.6° C. 36.5° C., or 36.4° C., or 36.3° C., or 36.2° C., or 36.1° C. 36.0° C. and to deploy the fan when the measured temperature exceeds, for example, 37.1° C., or 37.2° C., or 37.3° C., or 37.4° C., or 37.5° C., or 37.6° C., or 37.7° C., or 37.8° C., or 37.9° C., or 38.0° C.

Alternatively, it may be that the electronic circuit board 400 of the sensor card 20 performs temperature control on the sensor card 20 and simply sends a fan request and/or a pump request to the sensor card receiver 30 when the electronic circuit board 400 determines is necessary.

It may be also that the microfluidic cartridge is heated to approximately 37° C. prior to the application of blood in order not only to control a temperature of the blood near the microfluidic detection chambers but also to control a temperature of the blood throughout its journey through the microfluidic substrate. This may also assist with avoiding changes in blood viscosity due to temperature which may also contribute to increased reliability of travel of blood, buffer and blood-buffer mix within the microfluidic substrate 300, 500.

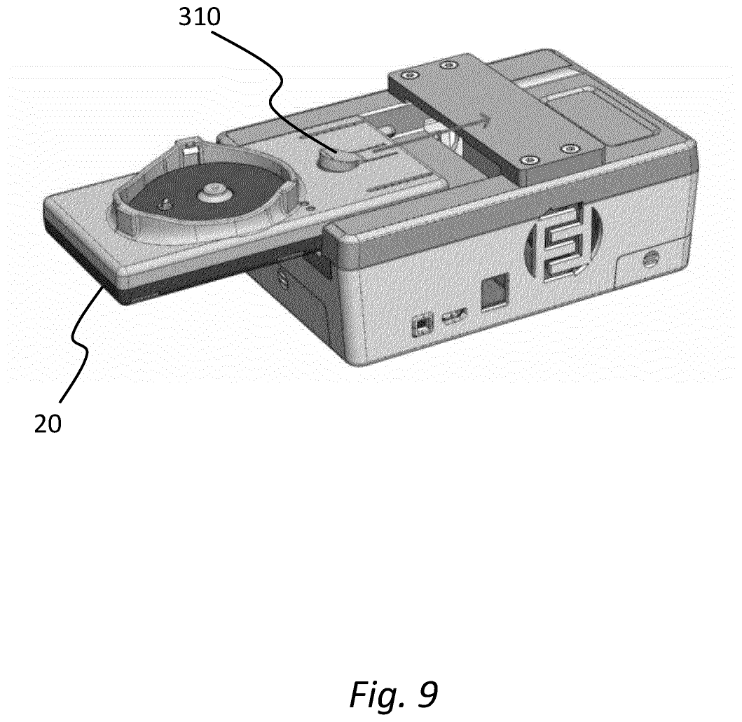

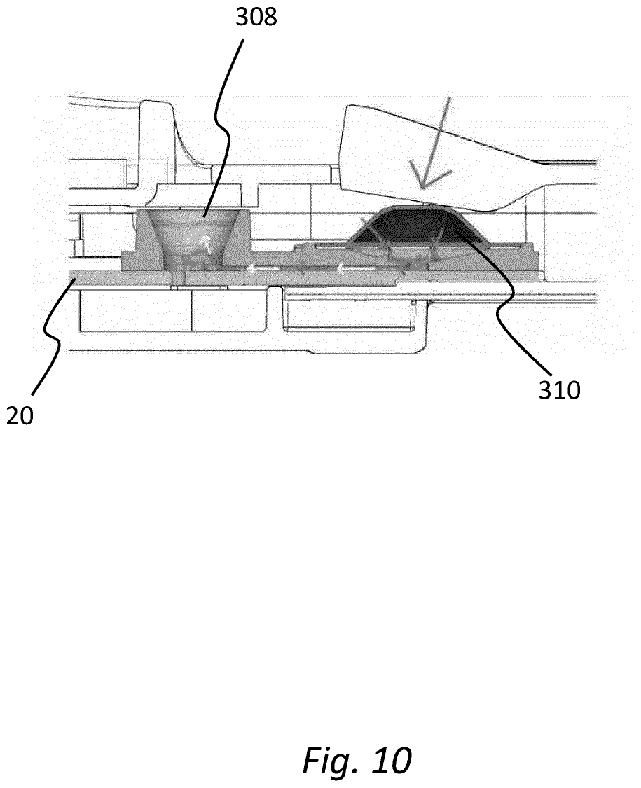

In use, the sensor card 20 is inserted into the sensor card receiver 30 in the manner shown in FIG. 9. Insertion of the sensor card 20 into the sensor card receiver 30 results in actuation of the buffer capsule trigger switch 311 (see FIG. 2) which pierces the buffer capsule 310 such that the buffer is released onto the microfluidic substrate 300, 500. The buffer capsule attached to microfluidic substrate 300, 500 located under the buffer capsule 310 may contain 150 μL of buffer, which may be filtered and/or degassed. For example, a gas release chamber 308 comprising a barrier may be provided immediately downstream of the buffer capsule 310 to separate air from the buffer, causing air to be released upwards to atmosphere and causing buffer to drop into the microfluidics.

Once blood has been received into the sample collection device 10, the sample collection device 10 is applied to the sensor card 20. Such that they come together as shown in FIG. 2.

Using the pump of the sensor card receiver 30, air may be injected from the sensor card receiver 30 into the sample collection device 10 in order to dislocate the capillary blood and push it out of the sample collection device 10 and into the microfluidic substrate 300, 500.

Once filtered, blood plasma diluted by the buffer may flow through the channels of the microfluidic substrate 300, 500. The diluted blood plasma may be divided into the requisite number of channels, which, in the illustrated embodiment, is 16 channels.

The sensor assembly 1 may be configured such that a chemical reaction which results in an emission of photos may or may not occur in some or all of the 16 microfluidic detection chambers. Therefore, by measuring photon emission in each of the 16 microfluidic detection chambers using the 16 primary arrays a pre-adjusted value indicative of light emitted within the corresponding microfluidic detection chamber may be obtained.

By sensing photons released during a chemical reaction (luminescence) rather than sensing photon emission from fluorescence or photon transmission following absorbance, a much larger dynamic scale can be measured. Furthermore, by contrast with fluorescence, there is no need for any emission of light to excite the fluorescent material, and by contrast with absorbance, there is no need to emit light to be selectively absorbed/transmitted.

Thus, there is no calculation (e.g. derivative) required in order to obtain a raw result for the number of photons emitted by the luminescent reaction, because every photon counts, and the result is obtained in real time. Moreover, for the same reasons, the dark count rate (obtained from the secondary arrays of photodiodes) is also obtained in real time.

However, in choosing to measure luminescence during a chemical reaction, there is only a single chance to measure the photons emitted during the reaction. This means there may be little or no opportunity to perform a preliminary analysis in order to determine the order of magnitude of the photon emission before deciding on the sensitivity (calibration) required of the measurement.

It is against this background that the solution of the present disclosure has been developed, involving (a) a plurality of small volume microfluidic detection chambers each coupled to a single primary array of single photon avalanche photodiodes; (b) so-called dark count analysis (described further below) so as to maximise the accuracy of the eventual results; and (c) careful temperature regulation to avoid variations due to fluctuating temperature. This final aspect may be particularly significant when aiming to reproduce in vivo conditions. One such example would be a blood coagulation assay.

With reference to dark count analysis, the sensor assembly 1 is further configured to use the dark count obtained from each secondary array of photodiodes to adjust the pre-adjusted value and thereby calculate for each sensing channel an adjusted value indicative of light emitted within the microfluidic detection chamber.

In one embodiment, where exactly half of the single photon avalanche photodiodes are primary array photodiodes and exactly half are secondary array photodiodes, the dark count rate may be normalised to account for any difference in area between the primary array photodiodes and the secondary array photodiodes, and may then be subtracted from the pre-adjusted value. Alternatively, this may be done on an average (e.g. mean or median) basis for the single photon avalanche photodiodes in the array. For example, the median value from the secondary array may be subtracted from the median value for the primary array. In addition, to the extent that any channels are determined to be operating outside expected parameters (which may suggest, for example, a photodiode fault) those channels are excluded from influencing the pre-adjusted value obtained from the primary array and the dark current adjustment obtained from the secondary array.

Using median photon counts across multiple channels performing the same reaction may be particularly useful for eliminating outlying results from photodiodes in an array that may not be functioning, or may not be functioning as expected.

In small scale device such as that set out in the preferred embodiment of the disclosure, small volume luminescent reactions may produce, at 37° C., a median count rate between 20,000 and 500 RLU (relative light units) in the primary photodiodes and, as such, the opacity of the covering on the secondary photodiodes needs to result in a dark count rate less than 10% of the sensing signal at the same temperature.

In some embodiments, rather than considering an absolute number of photons released in each microfluidic detection chamber (as determined from the adjusted value), it may be that a difference between the average (e.g. mean or median) result for different microfluidic detection chambers is considered, or a standard deviation.

For example, where a primary array comprises 64 photodiodes, a photon count may be taken for each of the 64 photodiodes, and where a secondary array comprises 16 photodiodes, a photon count may be taken for each of the 16 photodiodes. In order to eliminate results from photodiodes in each array that may be defective, a median result for the 64 primary array photodiodes may be considered as representative and a median result of the 16 secondary array photodiodes may be considered as representative. Thus, the median result for the secondary array may be subtracted from the median result for the primary array leaving an adjusted median value.

The disclosure is not limited to sensing samples of blood plasma and may be appropriate for detecting any luminescent (e.g. chemiluminescent) reaction in any small volume of liquid sample appropriate for the microfluidic channels of the sensor.

Although the illustrated embodiments have the microfluidic substrate and the semiconductor substrate present on the sensor card, with microprocessor functionality carried out on the sensor card receiver, in other embodiments the semiconductor substrate may be present on the sensor card receiver.

The reagents that might be used in the reaction reagent are not prescribed in the present specification. The first preliminary reagent (in the first preliminary reagent chamber (590)) may be the same as or different from the second preliminary reagent (in the second preliminary reagent chamber (690)). The first reaction reagent (in the first plurality of microfluidic detection chambers (710)) may be the same as or different from the second reaction reagent (in the second plurality of microfluidic detection chambers (720)). Appropriate reagents would depend upon the assays being conducted.

1. A sensor for sensing photons in liquids, the sensor comprising:
a microfluidic substrate comprising a blood inlet, a buffer inlet, a mixing region configured to mix blood with buffer to provide a sample, and a plurality of microfluidic detection chambers each configured to receive a volume of analyte of less than 5 micro litres, wherein the microfluidic substrate is non-transparent such as to prevent photons from passing through the microfluidic substrate, wherein the microfluidic substrate comprises a planar face and each microfluidic detection chamber comprises a transparent aperture located in the planar face;
a semiconductor substrate comprising a plurality of single photon avalanche photodiodes facing the planar face of the microfluidic substrate and arranged in a plurality of primary arrays of photodiodes and at least one secondary array of photodiodes,
a plurality of sensing channels, wherein each sensing channel of the plurality of sensing channels comprises one of the microfluidic detection chambers and a corresponding one of the primary arrays of photodiodes arranged such that each photodiode in the primary array is aligned with and no more than 1,000 micrometres from its corresponding microfluidic detection chamber so as to receive photons emitted within the corresponding microfluidic detection chamber, wherein a primary charge count derived from the primary array of photodiodes provides a pre-adjusted value of photon count indicative of a total number of photons emitted within the corresponding microfluidic detection chamber;
wherein each one of the photodiodes in the secondary array of photodiodes is covered to prevent ingress of light and wherein a secondary charge count derived from the secondary array of photodiodes is indicative of a dark count;
wherein an adjusted charge count is provided by using the dark count value to adjust the primary charge count.

2.-50. (canceled)

51. The sensor of claim 1, wherein the non-transparent microfluidic substrate has an optical density attenuation coefficient of at least 1,00, preferably at least 1,000.

52. The sensor of claim 1, wherein the microfluidic substrate further comprises a blood filter configured to receive blood from the blood inlet and to release blood plasma for mixing with buffer to provide a sample.

53. The sensor of claim 1, further comprising: a temperature sensor circuit comprising: a temperature sensor; a heating element; and a heat sink element for use in regulating a temperature of the material being sensed wherein optionally:
the temperature sensor circuit is one of a plurality of temperature sensor circuits and further optionally wherein each temperature sensor circuit of the plurality of temperature sensor circuits is located proximate to one or more primary arrays.

54. The sensor of claim 53, wherein each temperature sensor circuit of the plurality of temperature sensor circuits is located between a pair of adjacent primary arrays wherein optionally:
there is a one to one correspondence between each pair of primary arrays and each temperature sensor circuits such that there is a two to one correspondence between primary arrays and temperature sensor circuits.

55. The sensor of claim 53, wherein each temperature sensor circuit is configured to maintain a target temperature based on a difference between the target temperature and a value for measured temperature obtained from the temperature sensor, wherein optionally the target temperature is between 36° C. and 38° C., preferably 37° C.

56. The sensor of claim 1, wherein there is a one to one correspondence between each primary array of photodiodes and each secondary array of photodiodes.

57. The sensor of claim 53, wherein the temperature sensor circuit is configured to generate thermal energy in the heating element.

58. The sensor of claim 1, wherein the microfluidic substrate comprises an anti-fouling coating.

59. The sensor of claim 1, wherein:
each primary array of photodiodes comprises a grid of 64 single photon avalanche photodiodes; and/or
each secondary array of photodiodes comprises a grid of 16 single photon avalanche photodiodes; and/or
the plurality of primary arrays of photodiodes comprises 8 primary arrays and optionally the plurality of secondary arrays of photodiodes comprises 8 secondary arrays.

60. The sensor of claim 1, wherein the semiconductor substrate is mounted on a face of a printed circuit board, and wherein the printed circuit board is a single planar printed circuit board.

61. The sensor of claim 1, wherein the microfluidic substrate further comprises a blood filter configured to receive blood from the blood inlet and to release blood plasma for mixing with buffer to provide a sample and wherein the plurality of primary arrays of photodiodes comprises 8 primary arrays, wherein the temperature sensor, the heating element and the heat sink element are mounted on the printed circuit board.

62. The sensor of claim 1, wherein the semiconductor substrate is one of a pair of corresponding semiconductor substrates.

63. The sensor of claim 60, wherein the printed circuit board and the microfluidic substrate comprise corresponding alignment features for assisting alignment between each microfluidic detection chamber and its corresponding primary array of photodiodes.

64. The sensor of claim 1, wherein each one of the single photon avalanche photodiodes is configured to detect wavelengths within the visible spectrum.

65. The sensor of claim 1, wherein each one of the single photon avalanche photodiodes has a response time of less than 10 nanoseconds, preferably less than 1 nanosecond, more preferably less than 100 picoseconds; and/or
wherein each one of the single photon avalanche photodiodes has a dynamic range of at least four orders of magnitude.

66. The sensor of claim 1, further comprising a plasma separation membrane for separating red and white blood cells from plasma.

67. The sensor of claim 1, wherein each one of the photodiodes in the secondary arrays of photodiodes is covered to prevent ingress of light by a metal layer applied directly to the semiconductor substrate.

68. The sensor of claim 1, wherein the printed circuit board comprises an opaque coating across the face of the printed circuit board except in a region of the semiconductor substrate.

69. The sensor of claim 1, wherein the plurality of microfluidic detection chambers comprises a first subset of microfluidic detection chambers and a second subset of microfluidic detection chambers, and the microfluidic substrate comprises a junction configured to distribute a sample between the first subset of microfluidic detection chambers and the second subset of microfluidic detection chambers.

70. The sensor of claim 1, further comprising a controller configured to obtain the primary charge count and the secondary charge count and to calculate the adjusted charge count, wherein optionally the adjusted charge count is calculated by subtracting the secondary charge count from the primary charge count and/or wherein the adjusted charge count is calculated independently for each sensing channel.

71. The sensor of claim 71, wherein the controller is configured to compare the adjusted charge count for the first subset of microfluidic detection chambers with the adjusted count for the second subset of microfluidic detection chambers.

72. A sensor assembly comprising:
a sensor card comprising the sensor of claim 1; and
a sensor card receiver.

73. A method for sensing photons in liquids, the method comprising
providing a sensor that comprises:
a microfluidic substrate, wherein the microfluidic substrate is non-transparent such as to prevent photons from passing through the microfluidic substrate, the microfluidic substrate comprising a planar face and a plurality of microfluidic detection chambers each configured to receive a volume of liquid of less than 5 micro litres, wherein each microfluidic detection chamber comprises a transparent aperture located in the planar face;
a semiconductor substrate comprising a plurality of single photon avalanche photodiodes facing the planar face of the microfluidic substrate and arranged in a plurality of primary arrays of photodiodes and at least one secondary array of photodiodes,
a plurality of sensing channels, wherein each sensing channel of the plurality of sensing channels comprises one of the microfluidic detection chambers and a corresponding one of the primary arrays of photodiodes arranged such that each photodiode in the primary array is aligned with and no more than 1,000 micrometres from its corresponding microfluidic detection chamber so as to receive photons emitted within the corresponding microfluidic detection chamber, wherein a primary charge count derived from the primary array of photodiodes provides a pre-adjusted value of photon count indicative of a total number of photons emitted within the corresponding microfluidic detection chamber;
wherein each one of the photodiodes in the secondary array of photodiodes is covered to prevent ingress of light and wherein a secondary charge count derived from the secondary array of photodiodes is indicative of a dark count;
wherein the method comprises:
triggering a luminescent reaction to occur in the microfluidic detection chambers;
obtaining, for each sensing channel, the primary charge count;
obtaining, for each sensing channel, the secondary charge count; and
using the secondary charge count to adjust the primary charge count in order to calculate an adjusted charge count.

74. The method of claim 73, further comprising:
triggering a first luminescent reaction to occur in a first subset of the microfluidic detection chambers;
triggering a second luminescent reaction to occur in a second subset of the microfluidic detection chambers; and/or
wherein the primary charge count is a median charge count for the photodiodes in the primary array and wherein the secondary charge count is a median charge count for the photodiodes in the secondary array.
